(19) United States
(12) Patent Application Publication
(10) Pub. No.: US 2010/0043790 A1
(43) Pub. Date: Feb. 25, 2010

(54) NEBULISER VALVE

(76) Inventor: Andrew Tatarek, Hampshire (GB)

Correspondence Address: DRINKER BIDDLE & REATH (DC), 1500 K STREET, N.W., SUITE 1100, WASHINGTON, DC, 20005-1209, US

(21) Appl. No.: 12/310,401

(22) PCT Filed: Sep. 5, 2007
(86) PCT No.: PCT/GB2007/050524

§ 371 Date: Aug. 31, 2009

(30) Foreign Application Priority Data
Sep. 5, 2006 (GB) 0617417.1

(51) Int. Cl. A61M 16/20

(52) U.S. Cl. 128/203.14

(57) ABSTRACT

The nebuliser has a driving gas pathway through a nebulising section and the gas pathway includes a pneumatic control valve operable between an open configuration in which gas is able to flow from a supply inlet through the nebulising section to create an aerosol and to an airway with outlet for onward delivery and a closed configuration in which gas flow to the outlet is stopped. With the nebuliser drugs in an aerosol form can be delivered to a patient during the inhalation phase only and earlier in the inhalation cycle than can be achieved using conventional pneumatically activated nebulisers.

This invention relates to the field of nebulisers and, in particular, to a device that is operable to dispense a dose of a drug from the nebuliser during a limited part of a respiratory cycle.

Nebulisers are generally single-use low-cost devices. They are well known and used in a variety of applications to deliver drugs to patients. The drug or, rarely, drug mixture is poured in liquid form into the nebuliser just prior to treatment. When operated, a jet of gas is used to break the liquid into a fine mist or aerosol, which is then inhaled by a patient, typically via a mouthpiece or mask. Drug delivery by this method has two major benefits. First, the drug enters the bloodstream via the lungs, which is a faster route than oral administration and yet avoids the unpleasantness of needles and syringes. Secondly, airway drugs, such as bronchi-dilators are delivered directly to the site at which they are needed.

Conventional nebulisers are driven by a gas, either oxygen or air, supplied at a constant flow rate, sometimes via a flow meter. Typically the gas is drawn from an outlet pressurised to 1-4 bar and restricted to a flow of about 10 l/min. This flow rate may be provided either by means of a restriction at a high pressure source (e.g. 4 bar), for example a flow meter with a needle valve, or in the case of a low pressure source (e.g. 2 bar) by means of the nebuliser jet aperture. The constant flow of driving gas means that, in the conventional nebuliser, the aerosol is delivered to the patient continuously: the drug is dispensed regardless of whether the patient is inhaling or exhaling.

However, the patient can only make use of a drug that is dispensed during inhalation. The aerosol delivered during exhalation simply escapes to the atmosphere. This leads not only to unnecessary drug wastage, with consequent increase in nebuliser loading and cost, but also to a potential health hazard. Any drug that escapes from the nebuliser, or indeed that is exhaled by the patient, may negatively affect other persons in the vicinity.

The danger is not only limited to the escape of drugs: if the driving gas is oxygen, unused oxygen may be absorbed by clothing. This increases the flammability of the clothing, which is a known fire hazard both in homes and hospitals.

Some research has been directed towards development of a nebuliser that reduces the amount of aerosol escaping to the ambient air. Specifically, a nebuliser that dispenses aerosol during inhalation only has manifold advantages, including reducing wastage of the drug and reducing contamination of the surrounding environment.

A number of electronically-controlled nebulisers, which address this issue, are currently available. These however are not low-cost alternatives and consequently cannot compete with standard constant-flow nebulisers. They moreover suffer from the disadvantage of having to use batteries, leading to limited life.

U.S. Pat. No. 5,823,179 and U.S. Pat. No. 6,044,841 describe low cost nebulisers that operate pneumatically. Such nebulisers incorporate a movable gas diverter located in a chamber above the pressurised gas outlet and liquid outlet and separated by a variable height nebulising gap. The gas diverter is movable between a nebulising position in which pressurised gas from the gas outlet is directed across the liquid outlet to produce aerosol and a non-nebulising position in which the liquid outlet remains outside the gas flow. The diverter is moved in cycles in response to a patient's breathing: when the inhalation pressure exceeds the driving flow, the pressure under a piston or diaphragm is reduced and the diverter is pulled down to a position at which the aerosol is generated.

A problem with the existing art is its reliance on high inhalation flow to trigger aerosol release only once that flow exceeds that of the driving gas. Typically a flow rate of 8 l/min is used to drive a nebuliser and accordingly no aerosol will be delivered to a patient until inhalation flow exceeds this value. Even in a healthy person, this flow rate may not achieved until some time after the start of the inhalation cycle. Inhaled gas that reaches deep into the alveoli, and therefore transfers to the bloodstream, is primarily that taken at the start of a breath. The remainder fills the air passages and trachea and is then exhaled. It follows therefore that, for a drug to enter the bloodstream efficiently, it should be delivered to a patient at, or as soon as possible after, the start of inhalation. With the prior art pneumatic nebulisers the gas that reaches the alveoli may carry little or no aerosol. This problem is exacerbated for patients whose conditions cause them to take only shallow breaths. Furthermore, although the prior art does address the issue of drug wastage and contamination of ambient air by generating the aerosol during inhalation only, driving gas is still required to be supplied continuously. A compressed gas supply, such as a cylinder, will empty at the same rate as for a conventional nebuliser; there is no increase in its lifetime. Similarly, a battery powered compressor will be required to produce the same volume of compressed gas and the battery will last no longer than previously. It is also worth noting that the piston described in U.S. Pat. No. 6,044,841 must be manufactured to high tolerance in order to operate.

It is an object of the present invention to provide a nebuliser that is adapted so as not to deliver aerosol during the exhalation cycle. A further object of the present invention is to deliver aerosol promptly at the start of an inhalation cycle and either to deliver aerosol through the whole of the inhalation cycle or for a predetermined period of time from the start of the inhalation cycle.

The present invention provides a nebuliser comprising a driving gas pathway through a nebulising section characterised in that the gas pathway includes a pneumatic control valve operable between an open configuration in which gas is able to flow from a supply inlet through the nebulising section to create an aerosol and to an airway with an outlet for onward delivery and a closed configuration in which gas flow to the outlet is stopped.

This invention has the advantage that it is operable to deliver an aerosol for drug administration during selected time periods and to halt the driving gas supply, and hence drug delivery, otherwise. It accordingly lends itself to applications in which the aerosol is preferably delivered during patient inhalation only. Both drugs and gas will be conserved by inhibiting operation of the nebuliser when the patient is exhaling or after a predetermined time period from the start of a patient's inhalation. This is to be contrasted with pneumatic devices in which the gas flow is continuous. With the present invention, the control valve is preferably closed during exhalation, and consequently the driving gas, or the battery on a compressor, will not be wasted. Typically, the lifetime of a gas cylinder or battery powered compressor will have its lifetime extended threefold. A compressor used with the nebuliser of the present invention can have its capacity reduced by one third, and therefore can be made smaller, lighter and less expensive than previously.

The control valve is preferably located in the pathway between the inlet and nebulising section. As the pipes within the gas pathway are narrower than the airway, flow within the pipes is more readily controlled by a valve in comparison to controlling flow in the larger diameter of the airway.

The nebuliser may also include a sensing valve arranged to switch the control valve between its configurations in response to pressure variations in the airway. These pressure variations may be sensed using a sensing line which connects a sensing volume within the valve with an outlet in the airway. The airway pressure variations are preferably those arising during patient respiration.

The control valve may comprise a diaphragm separating a driving gas volume section of the driving gas pathway from a control volume wherein the diaphragm is moveable to seal an inlet to the driving gas volume in response to a rise in pressure within the control volume and to open the inlet to the driving gas volume in response to a fall in pressure within the control volume.

The sensing valve may comprise a diaphragm separating a switching gas volume from a sensing volume wherein the diaphragm is operable to seal a connecting passageway between the switching gas volume and the control volume of the control valve and to open said connecting passageway in response to a respective relative rise and fall of pressure within the sensing volume. The diaphragm is preferably sufficiently lightweight to be substantially unresponsive to the effects of gravity and forces acting on the sensing diaphragm are sufficiently balanced so as to permit it to be responsive to a change in pressure in the sensing volume arising from respiration.

The diaphragm may comprise a central portion and a peripheral portion, the central portion being more stiff than the peripheral portion. The central portion is preferably sufficiently stiff so as to exhibit minimal flexure in response to the forces generated by respiration whereas the peripheral portion is preferably sufficiently resilient to enable free movement of the central portion in response to the forces generated by respiration.

It is preferred that the diaphragm is in contact with a sealing member which, in one embodiment, may be adapted for movement into the passageway to seal the control volume.

The outlet of the sensing line, in one embodiment, intersects with the aerosol flow in the airway and the position of the outlet of the sensing line, relative to the nebulising section, may be selected in dependence on a desired difference in pressure between the pressure required to close the passageway and the pressure required to open the passageway.

The airway is preferably part of a user interface for application to the mouth and/or nose of a user and the user interface may include an outlet valve arranged to remain in a closed configuration unless gas flow from the nebulising section exceeds that being inhaled by the user. The user interface may also include an inlet valve which in one embodiment is a demand valve having means for connection to a gas supply.

The control valve may also include a restrictor in a passageway connecting the gas inlet with the control volume. In one embodiment, the restrictor is sized so as to ensure that the time taken to fill the control volume is less than 10% of the time of an average inhalation phase and, more preferably, is around 1 ms. In an alternative embodiment the restrictor is sized so as to ensure that the time taken to fill the control volume is in the range 0.2.-0.3 s.

The sensing valve may also include a restrictor which is sized such that the pressure acting on the sensing diaphragm required to seal the connecting passageway between the switching gas volume and the control volume of the control valve is more positive than that required to open said passageway.

Embodiments of the present invention will now be described by way of example only and with reference to the following drawings.

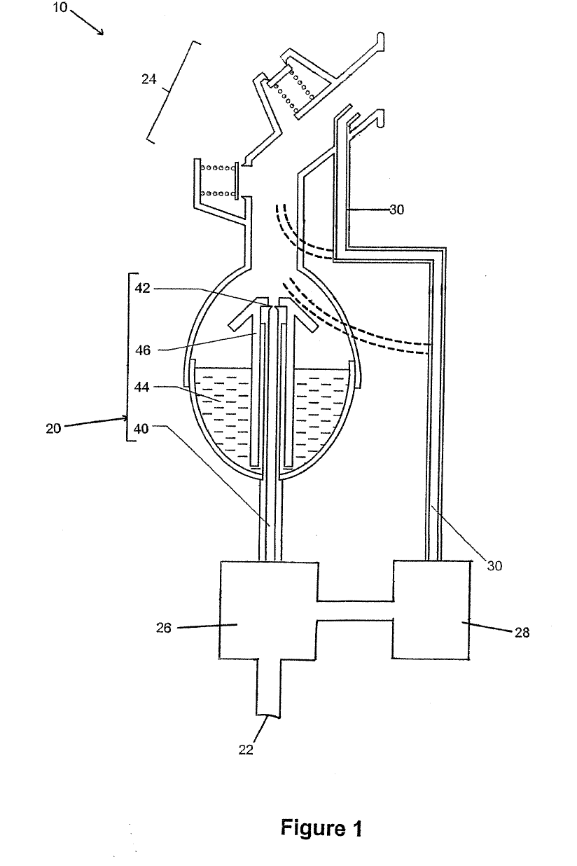

FIG. 1 is a schematic illustration of a design of a typical nebuliser in accordance with the present invention.

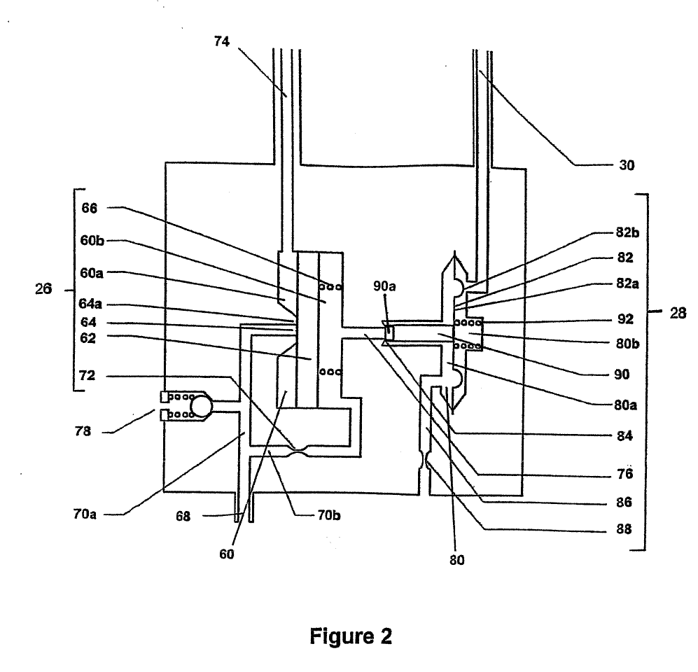

FIG. 2 is a schematic illustration of a first embodiment of a nebuliser control device in accordance with the present invention.

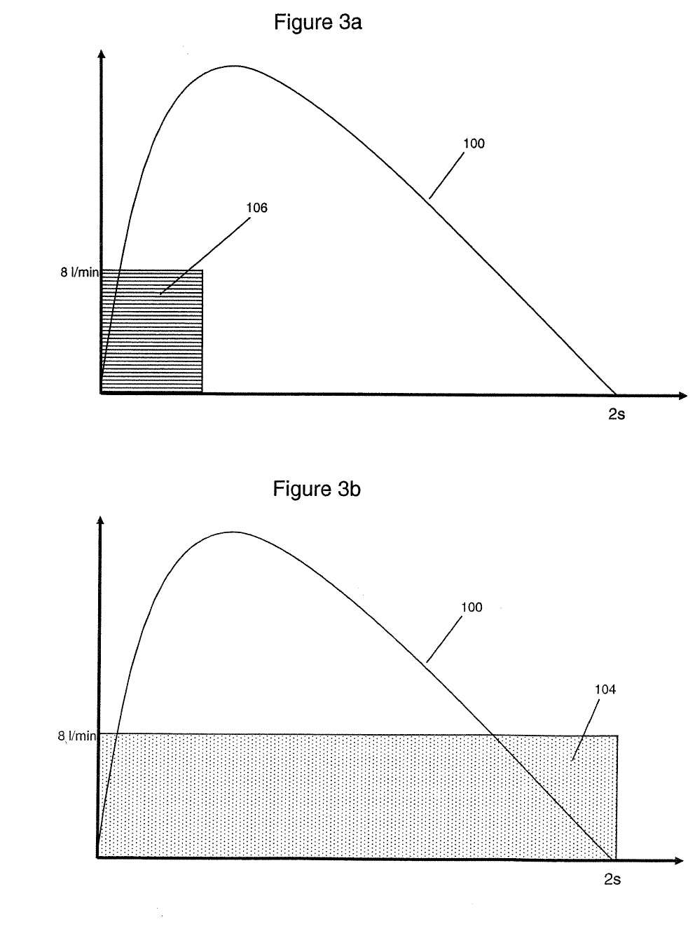

FIGS. 3a and 3b are graphical illustrations of the variation of flow rate with time during a typical inhalation (lasting typically 2 seconds), indicating two different aerosol delivery windows possible using nebulisers in accordance with the present invention.

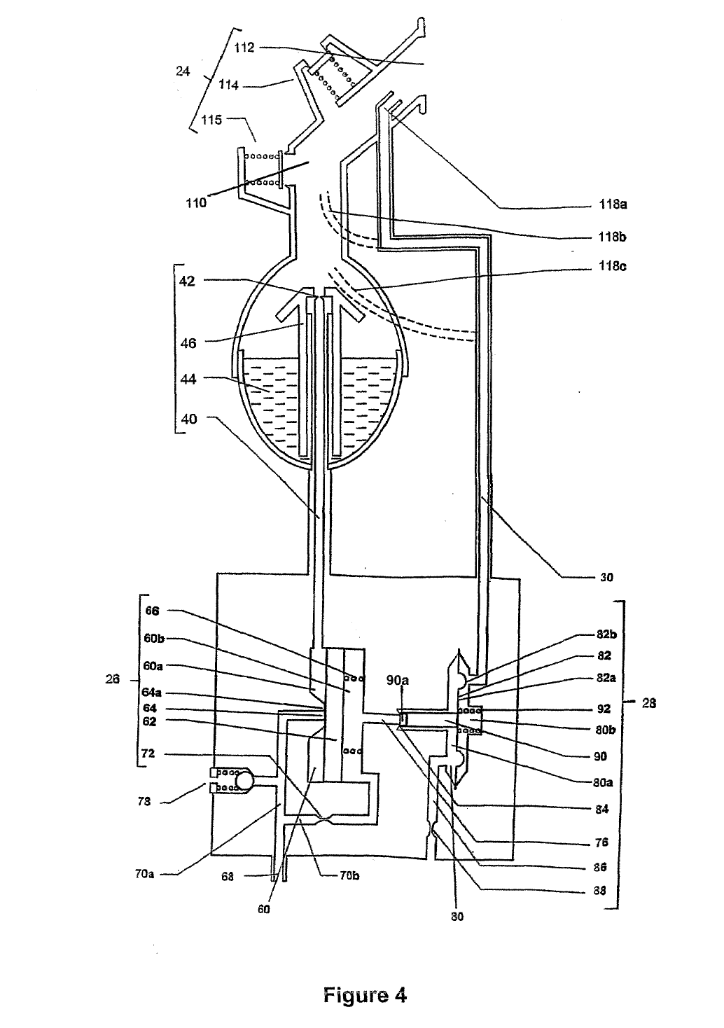

FIG. 4 is a schematic illustration of a nebuliser in accordance with the present invention.

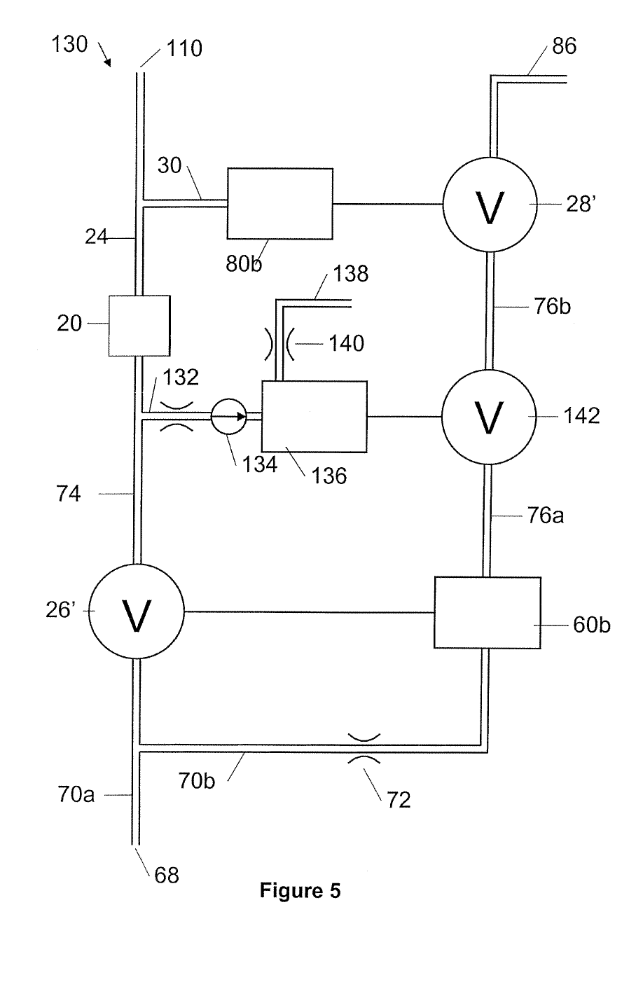

FIG. 5 is a block diagram illustrating a second embodiment of a nebuliser control device in accordance with the present invention.

Referring to FIG. 1, a nebuliser 10 in accordance with the present invention comprises a nebulising section 20 through which a driving gas passes to create an aerosol that is inhaled via a mouthpiece/patient interface 24. A control valve 26 is located between a gas inlet 22 and the nebulising section 20 to control the flow of the driving gas. The control valve 26 is switched by a sensing valve 28, which is in turn in fluid communication, via a sensing line 30, with the patient interface 24. The interface 24 may be a mouthpiece, mask to cover both nose and mouth, nasal cannula, or other such design that is commonly used with nebulisers. For convenience, a mouthpiece will be described in relation to this embodiment.

The nebulising section 20 is a standard design of jet nebuliser, which is well known, and so will be described only briefly in general terms. The invention may be applied to any type of jet nebuliser.

The nebulising section 20 comprises a gas inlet 40 through which the driving gas flows when the control valve 26 is open. The gas is formed into a jet as it is forced through an aperture 42. A drug to be dispensed is held in liquid form 44 within the nebulising section 20. The jet aperture 42 is located above the level of the liquid 44. A liquid drawing cap 46 draws the liquid 44 up to the vicinity of the jet, typically through capillary action and/or the pressure drop generated by the nebulising jet. The liquid 44 is then drawn from the cap 46 into the jet where it is broken into small particles, creating the aerosol. Typically, the aerosol flows at a rate of between 8 and 10 l/min (again, generally regulated either at supply or at the jet aperture) into the mouthpiece 24. When in use, the mouthpiece 24 is inserted into a patient's mouth for the aerosol to be inhaled by the patient.

When no driving gas pressure is present at the nebuliser inlet 40, no aerosol is delivered.

In this embodiment, the nebuliser is operated by switching on and off the driving gas pressure at the nebuliser inlet 40 in response to a patient's respiration cycle.

As the patient inhales, gas pressure within the mouthpiece 24 drops. This pressure drop is detected by the sensing valve 28 via the sensing line 30. The sensing valve 28 opens the control valve 26 and the driving gas passes through the nebulising section 20. As will be explained in more detail below, the sensing valve 28 and control valve 26 can be designed such that the control valve may be switched on very rapidly.

In one embodiment, the control valve 26 is kept open for a finite period of time, for example 0.2-0.3 s, and then closed. Closure of the control valve 26 prevents the driving gas passing to the nebuliser inlet 40 and so no aerosol is generated. The opening and closing of the control valve 26 is then repeated until the sending valve 28 ceases to sense inhalation. In an alternative embodiment the control valve 26 is kept open for a finite period of time and is then closed and only reopened in response to the start of a subsequent inhalation.

In a second embodiment, the sensing valve 28 is also arranged to detect a rise in pressure at the mask. This rise may be caused by the start of patient exhalation, or by a build up of aerosol gas as the inhalation flow rate falls below the gas driving rate of around 8 l/min. In either case, the sensing valve 28 is arranged to turn off the control valve 26 so that the driving gas is prevented from pressurising the nebuliser inlet 40.

Operation of the control valve 26 and the sensing valve 28 will now be described in more detail with reference to FIG. 2.

In the following description values of 5% and 95% of supply pressure are quoted as the trigger points for operation of the control valve. It should be noted that these are only nominal values given by way of example. In practice, 5% is in effect an abbreviation for a pressure just above atmospheric (or zero supply pressure) and 95% for a pressure just below the supply pressure. The actual values at which the valves trigger will depend upon the circumstances, such as the intended application of the nebuliser, and on the particular design of the valve elements.

The control valve 26 comprises a chamber 60 containing a control diaphragm 62, which is a disc of elastomeric material, whose outside diameter is sealed against the walls of the chamber 60. The chamber is therefore divided by the diaphragm into a driving volume 60a and a control volume 60b. A control valve jet 64 opens into the driving volume 60a. The diaphragm 62 is biased against the control valve jet 64 by a spring 66 such that the centre of the diaphragm 62 is sealably urged against a seat 64a of the jet 64. A gas inlet 68 for connection to a driving gas supply is in communication with the chamber 60 via first 70a and second 70b passageways. The first passageway 70a leads from the inlet 68 to the driving volume 60a via the jet 64. The second passageway 70b connects the inlet 68 via a restrictor 72 to the control volume 60b. An outlet passageway 74 leads from the driving volume 60a to the nebulising section inlet 40.

In operation, driving gas enters the nebuliser 10 through the inlet 68 at supply pressure, typically 1-4 bar. Known supply devices are, for example, the output from a medical pipeline system or regulator, the pressure of which varies depending on country or application. Alternatively, the supply device may be a liquid oxygen delivery system, typically regulated to a pressure of 1.5 bar, a compressed gas cylinder or battery-operated compressor.

When the diaphragm 62 is away from the seat 64a, driving gas passes along the first passageway 70a through the jet 64, driving volume 60a and outlet passageway 74 to the nebulising section inlet 40. The nebuliser 10 will therefore create an aerosol for delivery to a patient. With the diaphragm 62 in this open position, the nebuliser jet aperture 42 is substantially smaller than the effective opening of the jet 64 and so the gas pressure in the driving volume 60a and outlet passageway 74 is substantially the same as the supply pressure.

When the diaphragm 62 is sealably in contact with the seat 64a, the gas in the first passageway 70a is unable to pass between the jet 64 and the diaphragm 62 and so is blocked from reaching the nebulising section 20 and the patient. With the diaphragm 62 in its closed configuration, the remaining driving volume 60a is vented through passageways 74, 40 and nebuliser jet aperture 42 and so is at atmospheric pressure.

The thickness and shape of the diaphragm 62 and spring 66 are within a range to be determined by the operating parameters. The forces acting on and the stiffness of this diaphragm/spring assembly are such that a pressure of 95% of supply pressure in the control volume 60b is sufficient to overcome the supply pressure acting on the jet sealing area 64a and to cause the diaphragm 62 to seal against the seat 64a. Once the seal is made then the pressure in the control volume 60b will rise to the supply pressure via its communication 70b with the inlet 68. The restriction 72 within this second passageway 70b controls the flow rate into the control volume 60b and hence the time it takes to reach supply pressure.

The sensing valve 28 is arranged to vent the control volume 60b by opening connection passageway 76 in response to a patient's inhalation. The details of how this is achieved will be described in more detail later but, for the purposes of describing the operation of the control valve 26, it is suffice to simply state this effect. Once venting is begun, the pressure in the control volume 60b will fall. When this pressure falls to a level at which its resultant load on the area of the diaphragm 62 plus the biasing effect of the spring 66 is smaller than the supply pressure acting over the area of the jet seat 64a, the diaphragm 62 will move away from the seat 64a.

As the effective open area between the diaphragm 62 and the seat 64a (i.e. 2πr×d, where r is the radius of the annulus of the seat 64a and d the separation between the seat 64a and the centre of the diaphragm 62) approaches the area of the nebuliser jet aperture 42, the pressure in driving volume 60a, outlet 74 and nebulising section inlet 40 will rise very quickly to the level of the supply pressure. Thereafter, supply pressure in the driving volume 60a will be acting over the whole area of the diaphragm 62. The pressure in the control volume 60b on the other hand will have fallen close to atmospheric as its venting is controlled by the sensing valve 28. The central section of the diaphragm 62 will, accordingly, move rapidly into the control volume 60b.

The control valve 26 accordingly opens and the driving gas passes through to operate the nebulising section 20.

When the patient stops inhaling, the pressure in the patient interface or mouthpiece 24 will rise to atmospheric and above as the nebulising section 20 still delivers the aerosol jet. This rise in pressure is detected by the sensing valve 28, which causes a re-sealing of the control volume 60b. Detailed explanation of how this is achieved is unnecessary at this point and so will be addressed later. Once the control volume 60b is sealed, pressure will once again build up towards supply pressure as the escape route for the driving gas entering from the second passageway 70b and restrictor 72 is closed. Once the pressure in the control volume 60b reaches 95% of supply pressure, the diaphragm 62 is forced towards its seat 64a at the jet 64. As the diaphragm 62 approaches the seat 64a, it reaches a point at which the effective open area between the seat 64a and the diaphragm 62 is small compared to the area of the aperture 42 of the nebuliser jet. Gas in the driving volume 60a then escapes through the nebuliser aperture 42 faster than it is replenished through the jet 64, and the pressure falls rapidly (within around a millisecond, depending on the controlling volume and size of the restrictor in the venting line). The pressure in the driving volume 60a served to hold the diaphragm in its open position. As the pressure drops, so the control valve 26 closes rapidly, preventing driving gas flow to the nebulising section 20.

The control valve has now returned to its stable closed position.

The restrictor 72 controls the rate at which pressure builds in the control volume 60b once the sensing valve 28 is operated to close the control valve 26. In this embodiment it is simply a small orifice, which is sized so as to set the time between the pressure in the control volume 60b being at atmospheric (when the control valve is open) to 95% of supply pressure (when closing of the control valve diaphragm 62 is triggered), which is of the order 1 millisecond. Thus the total time for the control valve 26 to switch from an open to a closed configuration is the time taken to build up the pressure to 95% in the control volume 60b plus the time taken to reduce the pressure in the driving volume 60a to atmospheric. Both of these time periods can be set to be of the order of a millisecond.

A relief valve 78 is incorporated in the first passageway 70a from the gas supply inlet 68 to the jet 64. Such devices are well understood and so the design of the valve will not be described in further detail.

The relief valve 78 is not present in conventional nebulisers and is for use with a flow-meter supply, which is typically set at a pressure of 4 bar. A flow meter comprises a variable manually adjusted restriction and includes a flow indicator, typically a ball in a tapered tube with graduations on the side of the tube. It is typically connected to a nebuliser through a cuffed connection tube, which is designed to be connected and disconnected with light finger force.

In a conventional nebuliser the pressure in the connection tube is limited to the back-pressure of the nebuliser jet at the delivered flow rate, which is usually 1-2 bar, depending on nebuliser design. For the present invention however in which the nebulising jet is shut off by closing the control valve 26, the pressure in the connection tube 70a to the supply would almost immediately build up to the full 4 bar. Such a pressure is too high for standard connection tubes to withstand and unexpected and undesired disconnections can occur.

In such circumstances the relief valve 78 serves to limit the pressure in the line 70a to a level that the connection tube can withstand. Above the chosen trigger pressure of the relief valve 78 excess flow in the connection tube 70a is released to atmosphere.

Clearly, the detailed design of the parts of the control valve 26 will depend on operating parameters such as supply pressure, required nebulising flow, desired switching rate, etc. Suitable designs will be apparent to one skilled in the art once the functional requirements of the invention are appreciated. By way of example only an illustration of how an appropriate set of parts may be designed will be described.

Assume that the supply pressure is standard for a hospital system at 4 bar (0.4 Nmm⁻²). The nebulising section 20 is also of standard design with a requirement for an 8 lmin⁻¹ flow rate from this driving pressure.

In the control valve 26, an effective jet 64 diameter of, typically, 1.5 mm will permit 8 lmin⁻¹ flow with minimal pressure drop from 4 bar driving gas at the inlet 68. The load on the diaphragm 62 from the pressure at the jet 64 will be jet area×pressure=1.77 mm²×0.4 Nmm⁻²=0.71 N. In order to open the control valve 26, this pressure is required to overcome the biasing effect of the spring 66 and the closing force on the diaphragm 62 arising from the gas pressure in the control volume 60b. The spring force must therefore be less that the 0.71N exerted on the diaphragm 62 by the gas at the jet 64. A suitable design of spring would therefore have a load of, say, around half this value i.e. 0.35 N.

The diaphragm 62 may have an effective diameter of 5.5 mm, and hence an effective area of 23.8 mm². The forces (Fₒₚₑₙ) acting to open this are:
gas pressure acting over the area of the seat 64a (0.71 N)
gas pressure acting on the face around the seat

The forces (F_{close}) acting to close it are:
gas pressure in control volume 60b acting over the area of the diaphragm 62
spring or other biasing force (0.35 N).

The annular area of the face around the seat 64a is the effective area of the diaphragm 62 minus the area of the seat 64a=23.8−1.77=22.0 mm². If the gas pressure in control volume 60b is denoted P_{cv}, then two force balancing equations must be satisfied for the control valve 26 to be respectively closed and open.

If the control valve 26 is closed, the pressure in the annular area around the seat 64a is 0. Balancing opening and closing forces above:

Fₒₚₑₙ=0.71 N+0=F_{close}=23.8 mm²×P_{cv}+0.35 N=>P_{cv}=0.0149 Nmm⁻²=0.149 bar

If the control valve 26 is open, the pressure in the annular area around the seat 64a is the supply pressure of 4 bar. Balancing forces again:

Fₒₚₑₙ=0.71 N+22.0 mm²×0.4 Nmm⁻²=F_{close}=23.8 mm²×P_{cv}+0.35 N=>P_{cv}=0.385 Nmm⁻²=3.85 bar

As can be seen these values are close to the nominal trigger points of 5% and 95% of supply pressure respectively.

It will be noted that the contents of the control volume 60b are vented to atmosphere at the start of every breath. Accordingly, the smaller this volume 60b, the lower the wastage of driving gas.

It will be clear to one skilled in the art that many alternatives to the stated elements within the control valve 26 can be used. For example, the control diaphragm 62 could be replaced by equivalent sealing means, for example a piston with “o” rings. The biasing function supplied by the spring 66 could equivalently be provided by the diaphragm 62 located with its outside diameter to the left (as drawn in FIG. 2) of the top of the jet 64, again ensuring that the centre of the diaphragm 62 is sealably urged against the seat 64a; the urging force being similar to that provided by the spring 66.

In an alternative embodiment, the control valve 26 could be located in the airway of the patient interface 24, after the nebulising section 20. That is, it could be set to prevent aerosol reaching the patient after the aerosol is generated, rather than before. However, this embodiment requires the control valve 26 to be much larger as it is required to open and close the airway 110 in comparison to the much narrower gas supply pipe 70a of the first embodiment. This also makes this alternative embodiment more wasteful of the driving gas and all elements of the nebulising section 20 have to withstand the full supply pressure. Aerosol at pressure is therefore permitted to build up in a larger volume which may enable an initial “burst” release of drug each time the valve 26 is opened, which could be advantageous.

As explained above, the control valve 26 is switched in response to a patient's inhalation or exhalation. However, the forces involved in closing and opening the driving gas jet are far larger than those that could be produced by respiration pressures acting on a diaphragm of a practical size. Accordingly, some form of servo system for controlling the control valve 26 is preferred. In this embodiment of the invention, a second valve system, the sensing valve 28, is employed.

The sensing valve 28 comprises a chamber 80 containing a very light sensing diaphragm 82. For example, the weight of the diaphragm 82 is desired to be as small as possible in comparison to the forces acting on it so as to minimise external effects such as gravity and vibration: the weight of the diaphragm 82 typically may be in the region of 0.2-0.5 g. The diaphragm 82 is sealed across the chamber 80 dividing it into two parts: a switching volume 80a and a sensing volume 80b. The connecting passageway 76 from the control volume 60b within the control valve 26 opens into the switching volume 80a through a venting jet 84. The switching volume 80a is also open to atmosphere via a passageway 86 containing a restrictor 88. The diaphragm 82 comprises a relatively stiff central portion 82a and a resilient peripheral portion 82b that allows the central portion 82a to move freely in a direction perpendicular to its face. A sealing member 90, in the form of a cylindrical element, is located adjacent the central portion 82a of the diaphragm on its switching volume 80a side. The sealing member 90 has a resilient surface 90a and is moveable so as to seal the venting jet 84 by means of the resilient surface 90a. The diaphragm 82 is biased towards the sealing member 90 by a spring 92 or other suitable biasing means. The biasing load is such that the sealing member 90 seals against the venting jet 84 when the control volume 60b is at supply pressure. That is, the force of the spring 92 overcomes the force of the supply pressure acting over the area of the venting jet 84 as well as other external forces, including gravity, acting on the sealing member 90 and the diaphragm. The inside of the patient interface 24 is in fluid communication with the sensing volume 80b via the sensing line 30.

If both the switching volume 80a and sensing volume 80b are connected to atmospheric pressure by means of their connecting passageways 86, 30, then there will be zero pressure difference across the diaphragm 82. In this state, the biasing effect of the spring is sufficient to seal the sealing member 90 against the venting jet 84. The control volume 60b cannot then vent to atmosphere, supply pressure is maintained and the control valve 26 is shut.

If however the patient interface 24 attached to the nebuliser is placed over the mouth and/or nose of a patient, the pressure in the interface 24 will fall as the patient starts to inhale. This pressure drop is communicated down the sensing line 30 to the sensing volume 80b. If the pressure in the sensing volume 80b falls sufficiently to overcome the spring bias and diaphragm stiffness, the diaphragm 82 will move away from the sealing member 90. The sealing member 90 will then be pushed away from the venting jet 84 by pressure acting from the control volume 60b over its seat and so the seal will be opened. Gas in control volume 60b will flow past the sealing member 90 and escape through passageway 86 to atmosphere. When the pressure in the control volume 60b falls to 95% of the supply pressure, the control valve diaphragm 62 will move away from the seat 64a of the jet, opening the valve 26 and allowing driving gas to enter the nebulising section 20. Aerosol will then be released for inhalation by the patient.

If the pressure in the mouthpiece 24 starts to increase, for example as a result of the patient exhaling, or a build up of aerosol as inhalation stops, the rise to atmospheric pressure and above is communicated down the sensing line 30 to the sensing volume 80b. The resultant force over the area of the sensing diaphragm 82 combined with the force of the spring 92 biasing it, pushes the diaphragm 82 back towards the switching volume 80a. The diaphragm 82 in turn urges the sealing member 90 onto the venting jet 84 to re-establish the seal. Gas escape from the control volume 60b is prevented, the pressure of the control volume 60b returns to supply pressure, the control valve closes and switches off the driving gas to the nebulising section 20.

The pressure drop in the sensing volume 80b required to open the seal with the control volume 60b is termed the “cracking pressure”. It is likely to be of the order of 1-20 mm H₂O, according to the intended application and design of the device. The sensing valve 28 is required to be responsive to relatively small pressure changes and thereby to effect a more significant pressure change in the control valve 26. The small forces involved in operation of the sensing valve 28 mean that the lightest possible diaphragm 82 is preferred, the venting jet 84 should be small and the sealing member 90 should be able to seal against the jet 84 with very little force.

It will be recalled that the control volume 60b within the control valve 26 is required to vent almost instantaneously as the control diaphragm 62 moves away from the supply gas jet 64. For this reason, the open area of the venting jet 84 should be large compared with the size of the control restrictor 72. This allows greater gas flow out of the control volume 60b in comparison to flow through the restrictor 72 which enables the control volume 60b to vent close to atmospheric pressure when venting jet 84 is open.

In an alternative embodiment, the sealing member 90 is absent and the diaphragm 82 includes a resilient disc by which it is arranged to seal the venting jet 84 directly. This is however not preferred as there are known issues with leakage arising as the diaphragm seal does not move squarely with the jet 84. The increased length of engagement provided by the sealing member 90 increases the chance of squareness of the seal to the jet 84, which in turn reduces the force required to effect the seal.

Referring now to FIGS. 3a and 3b, a graph 100 of flow rate (y axis) against time (x axis) for a typical inhalation pattern is shown. The relative flow rate of 8 lmin⁻¹, which is typical of nebuliser designs, is also indicated along the y axis. This value is used for illustrative purposes only and is not to be seen as limiting the nebuliser flow rate for use with this invention in any way. At the start of an inhalation, the flow rate will be below the rate at which the nebulising section 20, when operating, ejects aerosol into the mouthpiece 24. Inhalation flow rate then increases to a maximum value (e.g. typically 20-50 lmin⁻¹) and then drops back to 0, after typically 2 s, in preparation for the start of exhalation. In FIG. 3b shaded area 104 indicates the ideal flow rate pattern for aerosol production using a nebuliser 10 in accordance with this embodiment of the invention. Gas flow (and hence aerosol delivery) will be rapidly started in the nebuliser 10 at the onset of inhalation, continue at a steady rate of 8 lmin⁻¹, and switch off rapidly when inhalation flow rate returns to 0. The nebuliser 10 remains off throughout exhalation, being arranged to start delivering aerosol only when switched on in response to the pressure drop in the mouthpiece 24 at the start of the following inhalation. Obviously practical nebulisers will show some deviation from this, whether by design or otherwise. For example, aerosol delivery at only the start of inhalation may be efficacious in some circumstances. The nebuliser 10 may therefore be arranged to turn off sooner, for example after around 0.5 s after the start of an inhalation.

An alternative design of nebuliser in accordance with this invention will provide aerosol generation with a flow rate profile 106 indicated by the horizontally shaded area of the graph in FIG. 3a. Details of this design will be described in relation to FIG. 5 below. Aerosol generated by this means however is present only at the start of the inhalation. This will conserve more driving gas and may be preferred for drug delivery to the bloodstream: only aerosol taken at the start of an inhalation will reach the alveoli and thereby deliver active agents to the bloodstream. By way of contrast the profile 104 of drug delivery in accordance with the embodiments of the invention described in relation to FIGS. 2 and 4 provides for continuous delivery to the lungs during inhalation. Drugs intended to have an effect both on the alveoli and airways within the lungs are better delivered by this method.

A problem with the design of the nebuliser shown in FIGS. 2 and 4 can be appreciated with reference to FIG. 3b. It is evident that as aerosol flow starts, if the patient is inhaling more than the 8 lmin⁻¹ that the nebulising section 20 is supplying then the negative (i.e. below atmospheric) pressure in the sensing volume 80b will be maintained. The sensing diaphragm 82 will remain in the open position and so will the control valve 26. Aerosol supply will thus be maintained.

On the other hand, if the patient is inhaling less flow than the nebulising section 20 is supplying, then pressure will build within the mouthpiece 24. Once it rises above atmospheric, the sensing diaphragm 82 will be forced back to its sealing position, closing the control valve and shutting off the aerosol generation. The pressure within the mouthpiece 24 will then start to fall again, and the aerosol restarted, and so on. This continuous “hunting”—partial opening and closing, will continue throughout the initial phase of inhalation, until the patient's flow exceeds 8 lmin⁻¹ or stops. Until this point, delivery of the aerosol is compromised.

Fuller details of the design of the nebuliser 10 described in relation to FIG. 2 are shown in FIG. 4. In this Figure, common elements are referenced as before. Gas is driven via the inlet 22 through the nebulising section 20 to create an aerosol that is inhaled via the mouthpiece/patient interface 24. The control valve 26 controls flow of the driving gas and is switched by a sensing valve 28. The sensing valve 28 is responsive to pressure changes at the patient interface 24, which are communicated to it via the sensing line 30. Adaptations to the mouthpiece 24 to improve the performance of the nebuliser 10 will be described in relation to this Figure.

The mouthpiece 24 comprises an airway 110 extending from the nebulising section 20 to an outlet 112 for positioning at a mouth. Inlet 114 and outlet 115 valves allow fluid flow into and out of the airway 110 respectively. The sensing line 30 extends from the sensing volume 80b within the sensing valve 28 to an outlet 118a within the airway 110. The outlet 118a lies in the path of the aerosol flow from the jet 42 of the nebuliser. Alternative outlet positions 118b, 118c within the airway 110 are also within the path of the nebuliser flow.

As observed previously in relation to FIG. 3b, the nebulising section 20 delivers aerosol to the airway 110 at a substantially constant flow rate. The flow rate during inhalation on the other hand varies over a breathing cycle and may be above or below the aerosol delivery rate. If the inhalation rate exceeds the aerosol flow rate, the inlet valve 114 opens to allow ambient air to flow into the mouthpiece 24 and supplement the aerosol flow. The inlet valve 114 will close again once the inhalation rate falls below the aerosol rate. If the aerosol flow rate exceeds the inhalation rate, which will result in an increase in pressure within the airway 110, the outlet valve 115 will open, when the increase in pressure exceeds a predetermined threshold, to allow excess aerosol to flow out to ambient air. The outlet valve 115 will close again once the inhalation rate rises. The inlet 114 and outlet 115 valves may be of any suitable construction, for example a sprung valve, a flap valve or a plain orifice.

The positioning of the outlet 118a, 118b, 118c of the sensing line 30 within the airway 110 is important if it is to be used to solve the problem of hunting referred to above. That is, the outlet 118a, b, c is placed in the path of the aerosol flow from the jet 42 of the nebuliser. In this position, once the nebuliser flow begins, a venturi effect reduces the pressure at the outlet 118a, b, c as gas is drawn from the sensing line 30 and the pressure in the sensing volume 80b is maintained below atmospheric. The control valve 26 remains open and the nebuliser flow is uninterrupted. Excess aerosol escapes through the outlet valve 115, until the patient is inhaling more flow than delivered by the nebulizer 10.

In making use of the venturi effect however, the negative pressure caused by this effect must be overcome before the sensing valve 28 will operate to close the control valve and shut off the nebulising flow.

Once the inhalation rate begins to fall below the aerosol rate, as it will do as inhalation comes to its end, the pressure will again increase within the airway 110. This increasing pressure can be used to counter the venturi effect. The balance point between the exhalation valve 115 and the venturi effect can be set such that, for example, the nebulising flow is shut off if the inhalation flow from the patient falls below, say, 1 lmin⁻¹. Alternatively, the relevant parameters may be set to specify a higher pressure requirement that needs a small degree of exhalation from the patient before the control valve is closed. In any case, the outlet valve 115 is designed such that the size of the open area and pressure required to open it prevent opening until after the control valve 26 is switched off.

An alternative method to create a differential pressure in the sensing volume 80b, such that a higher pressure is required to close it than to open it (and so prevent rapid switching between on and off), is provided by adding the restriction 88 to the switching volume's vent 86 to atmosphere. When the sensing valve 28 is open, gas from the supply will flow through the control valve restrictor 72 to the control volume 60b, which is vented via the switching volume 80a, passageway 86 and sensing restrictor 88. The restrictors 72, 88 are set such that the pressure in the switching volume 80a builds to slightly above atmospheric. This characteristic means that the pressure within sensing volume 80b required to close the sensing valve 28 is less negative than that required to open it. It may even be set such that the sensing volume 80b requires a positive pressure before the sensing valve closes.

This balance of restrictors may be used in combination with the mouthpiece outlet valve 115 to achieve full opening of the control valve 26 even if the patient is drawing less flow than the nebulising section 20 is delivering.

The pressure required to close the control valve 26 can also be balanced against the mouthpiece outlet valve 115 to set the sensing valve 28 to close at the end of an inhalation or at the start of an exhalation. That is, the nebulising section 20 will continue to deliver aerosol when the patient is drawing less than it is delivering, to a very low flow.

A flap valve, sprung valve, or the like (not shown) is incorporated in the airway 110 downstream of the sensing line 30 in order to ensure that the nebulising section 20 will cease to deliver aerosol if the mouthpiece 24 is removed from the patient's mouth while inhaling.

Clearly, it is not necessary to have the outlet 118 of the sensing line 30 within the aerosol flow path in this embodiment of the invention. It simply has to be within the airway 110 to permit the sensing valve 28 to detect a pressure change.

In another embodiment, also designed to overcome the problem of rapid turning on and off, the control valve restrictor 72 is a smaller sized orifice than provided in the previous embodiments. A smaller orifice 72 will increase the time it takes for the pressure in the control volume 60b to rise from atmospheric (when the control valve is open) to 95% of supply pressure (when the closed configuration of the control valve diaphragm 62 is triggered). In this embodiment, this time is set to be between 0.2 and 0.3 s.

When the patient starts to inhale, the control valve 26 opens, as described previously. Thereafter, regardless of the patient's inhalation rate or of the pressure in the sensing volume 80b, the control diaphragm 62 will remain open until the pressure in the control volume 60b reaches 95% of supply pressure. That is, until 0.2-0.3 s have elapsed. During inhalation therefore, a nebulising section 20 designed in accordance with this embodiment will deliver a series of short (0.2-0.3 s) pulses of aerosol flow. If a pulse is initiated close to the end of an inhalation breath then it will extend into the exhalation for the time delay. This delay time is therefore a compromise between minimising disruption to the aerosol supply arising from the stop-start pattern when inhalation is less than nebuliser flow rate and minimising wastage by extending aerosol delivery into the patient's exhalation.

It will be apparent to one skilled in the art that, regardless of the particular embodiment, the parts of the invention can be arranged to make the size of the control volume within the control valve almost insignificant. The gas consumed by the device will therefore be very small—less than 1% of the delivered flow is achievable.

The sensing line 30 and connections 40, 74 between nebulising section 20 and control valve 26 can be sufficiently long to allow the nebulising section 20 to be used remotely from the valves 26 and 28 e.g. typically 1.5 m long and/or where the nebulising section 20 is intended for single use application and is removably attached to the valves 26, 28 which are a permanent fixture. The volume in the lines 40, 74 should however be kept small, e.g. around 1 mm diameter, as the compressed gas inside the pipes remaining after the control valve 26 closes is vented to atmosphere. A large volume of remaining gas in the lines 40, 74 would significantly extend the time required to vent and thereby prolong aerosol delivery into the exhalation cycle.

FIG. 5 shows an alternative embodiment of valve system 26, 28 for use with this invention. This valve system 130 is of a type described in detail in WO 2006/092635 and is designed to deliver a single pulse of gas at the start of an inhalation and to delay sensing of a triggering pressure (e.g. inhalation pressure) for a set time period. That is, it provides an alternative means to prevent re-opening of the control valve 26 in response to a pressure change stimulus when such re-opening is undesirable. With this embodiment the delay is set to extend at least for the remaining duration of the inhalation and may extend into the exhalation phase. Following this delay the valve system is reset to a steady state in readiness for activation by the start of a subsequent inhalation.

This FIG. 5 uses a different representation than previously, but like components are similarly referenced. The slight exception is the collective reference to the control valve 26 and sensing valve 28. Each component has, for ease of reference, its control volume 60b, 80b, illustrated separately, outside of each valve assembly. Accordingly, numerals 26′ and 28′ will be used to denote the diaphragm, switching volume, etc. that remain once the control volume has been separately considered.

The control valve 26′ controls driving gas flow from the inlet 68 to the outlet 74 to the nebulising section (not shown). The control valve 26′ is controlled by the level of pressure in the main control volume 60b: when this pressure rises to around 95% of supply pressure, the valve 26′ closes and when it falls below around 5% of supply pressure the valve 26′ opens.

The main control volume 60b is pressurised from the input 68 via the passageway 70b containing the restrictor 72. The flow through the restrictor 72 is set such that the pressure build up in the main control volume 60b from a “flow on” condition to a “flow off” condition is the time for which flow is required—i.e. the amount of time from the start of a breath to give the ideal dose from the nebuliser.

The nebulising section 20 is in communication with the gas supply line 74 after the main control valve 26′.

The device 130 is triggered by negative pressure sensed in the sensing volume 80b connected via a sensing line 30 to the patient interface 24. The level of pressure in the sensing volume 80b controls the sensing valve 28′, which allows air from the main control volume 60b to vent to atmosphere as illustrated, via the venting passageway 86. When the pressure in the main control volume drops to a sufficient level, the control valve 26′ is opened to start flow to the patient. Immediately the control valve opens, the pressure in the sensing volume 80b rises, which closes the sensing valve 28′ and stops the venting of the main control volume 60b. From this moment, the pressure in the main control volume 60b goes up, fed from passageway 70b and the restrictor 72, until the level of pressure in the main control volume 60b reaches a sufficient level to close the control valve 26′ and cut off the flow to the airway 110.

The invention described in WO 2006/092635 addresses the fundamental problem that, as a result of the flow stopping, there is no longer an elevated pressure in the mouthpiece 24 to keep the sensing valve 28′ closed. Therefore, if at this moment the patient is still inhaling, the sensing valve 28′ opens again, and the main control volume 60b vents, thus opening the control valve 26′ again to deliver another pulse of driving gas. This second pulse of driving gas is not dispensed at an ideal point during inhalation and therefore is likely to mainly go to waste.

In order to prevent the sensing valve 28′ re-opening the control valve 26′, the assembly 130 also contains a mechanism for inhibiting its response to the drop in pressure as the nebulising flow is stopped. This mechanism is arranged to operate for a predetermined period following delivery of a pulse of driving gas to the nebulising section 20.

In the embodiment shown in FIG. 5, operation of the sensing valve 28′ is inhibited by further components connected as follows. Branching from a section of the output line 74 is a passageway 132 including a one-way valve 134 connecting to a sensing delay volume 136. The valve 134 is such as to allow pressurising of the volume from the line 74, but not flow in the reverse direction. The volume is vented by a vent line 138 including a flow restrictor 140. The vent line may vent to atmosphere, or to some other suitable point, for example, the patient interface 24. The drawing illustrates a vent to atmosphere.

The restrictor 140 is set such that the time taken for the volume 136 to vent from supply pressure to 5% of supply pressure is a predetermined period for which it is required to delay normal functioning of the sensing valve 28′—in other words, the period from the termination of the delivery pulse of gas to the nebulising section 20 to a point in time during the subsequent exhalation.

The level of pressure in the sensing delay volume 136 controls the operation of a sensing delay valve 142, which is connected in the vent line 76 from main control volume 60b, thus dividing the vent line into a first section 76a between the volume 60b and the valve 142 and a second section 76b between the valve 142 and the sensing valve 28′. The valve 142 is normally open, corresponding to the situation in which the pressure in the sensing delay volume 32 is less than 5% of the supply pressure. The valve is set such that it closes when the pressure in the sensing delay volume rises above 5% of supply pressure.

With this arrangement, once the pulse of gas has been delivered to the nebuliser, and hence aerosol to the patient, operation of the sensing valve 28′ is inhibited so that it is unable to reactuate the main control valve 26′ to supply another pulse. Generally speaking the timings will be such as to keep the main control valve 26′ open typically for about half a second, this period being known to provide sufficient aerosol to the patient under normal circumstances. This period could however be changed, as needed to suit the application. Following delivery of the pulse of gas, the operation of the sensing valve 28′ is inhibited for a predetermined period in order to prevent further gas flow. This predetermined period may be set to start and end at various different times, but should at least include that part of the expected inhalation of the user which follows the end of the pulse of gas. In a preferred embodiment, the predetermined period starts at the termination of the pulse, and terminates at a time after the end of the inhalation period which caused the delivery of the pulse of gas, but before the commencement of the next inhalation period—in other words, at a time during exhalation. If the predetermined period commences at the time that the main control valve closes to terminate the delivered pulse of gas to the user, then the predetermined period is likely to be typically about 1.5 seconds.

If the valve assembly of FIG. 5 is employed in the present invention, then a single pulse of driving gas may be administered at the start of inhalation, as shown in horizontally striped area 106 in FIG. 3a, and so does not deliver the aerosol across the remainder of the inhalation. The drug delivered at the onset of inhalation is important for uptake to the bloodstream, but other drugs such as bronchi-dilators may be more preferably delivered to both airways and alveoli.

It will be appreciated that details of the embodiments described herein may be changed without departing from the invention as set out in the accompanying claims. For example, inhalation valve 114 may be replaced with a demand valve connected to a separate gas supply so that the gas inhaled above that delivered by the nebuliser may be, for example, oxygen. Also, the diaphragms of the control valve 26 and the sensing valve 28 may be replaced for example with pistons and associated ‘o’ rings.

Furthermore, the venting jet 84 may be substituted with an aperture into which a pin, replacing sealing member 90, projects such that the conical surface of the pin forms a seal with the contacting edge of the aperture.

With the present invention drugs in an aerosol form can be delivered to a patient during the inhalation phase only and earlier in the inhalation cycle than can be achieved using conventional pneumatically activated nebulisers.

1. A nebuliser comprising a driving gas pathway through a nebulising section wherein the gas pathway includes a pneumatic control valve operable between an open configuration in which gas is able to flow from a supply inlet through the nebulising section to create an aerosol and to an airway with an outlet for onward delivery and a closed configuration in which gas flow to the outlet is stopped.

2. A nebuliser according to claim 1 in which the control valve is located in that portion of the gas pathway between the supply inlet and the nebulising section.

3. A nebuliser according to claim 1 further comprising a sensing valve arranged to switch the control valve between its configurations in response to pressure variation in the airway.

4. A nebuliser according to claim 1 wherein the control valve comprises a diaphragm separating a driving gas volume section of the driving gas pathway from a control volume and wherein, the diaphragm is moveable to seal an inlet to the driving gas volume in response to a rise in pressure within the control volume and to open the inlet to the driving gas volume in response to a fall in pressure within the control volume.

5. A nebuliser according to claim 4 wherein the sensing valve includes a sensing line having an outlet located in the airway, the sensing line connecting a sensing volume within the sensing valve with the outlet.

6. A nebuliser according to claim 5 wherein the sensing valve comprises a sensing diaphragm separating a switching gas volume from the sensing volume, the sensing diaphragm being operable to close a connecting passageway between the switching gas volume and the control volume of the control valve in response to a rise of pressure within the sensing volume and to open said connecting passageway in response to a fall of pressure within the sensing volume.

7. A nebuliser according to claim 6 wherein the sensing diaphragm is sufficiently lightweight to be substantially unresponsive to the effects of gravity and the forces acting on the sensing diaphragm are sufficiently balanced so as to permit it to be responsive to changes in pressure in the sensing volume arising from respiration.

8. A nebuliser according to claim 7 wherein the diaphragm comprises a central portion and a peripheral portion, the central portion having a stiffness greater than the peripheral portion.

9. A nebuliser according to claim 7 wherein the sensing diaphragm is in contact with a sealing member which is moveable to seal the control volume.

10. A nebuliser according to claim 7 wherein the sensing diaphragm is in contact with a sealing member which is movable within the passageway to seal the control volume.

11. A nebuliser according to claim 5 wherein the outlet of the sensing line intersects with the flow of aerosol from the nebulising section within the airway.

12. A nebuliser according to claim 11 wherein the position of the outlet of the sensing line relative to the nebulising section is selected in dependence on a desired difference in pressure between the pressure required to close the passageway and the pressure required to open the passageway.

13. A nebuliser according to claim 1 in which the airway is included in a user interface for application to the mouth and/or nose of a user and wherein the user interface includes an outlet valve arranged to remain in a closed configuration unless gas flow from the nebulising section exceeds that being inhaled by the user.

14. A nebuliser according to claim 13 wherein the user interface also includes an inlet valve.

15. A nebuliser according to claim 14 wherein the inlet valve is a demand valve having means for connection to a gas supply.

16. A nebuliser according to claim 4 wherein the control valve includes a restrictor in a passageway connecting the gas inlet with the control volume.

17. A nebuliser according to claim 16 wherein the restrictor is sized so as to ensure that the time taken to fill the control volume is substantially 10% of an average inhalation phase.

18. A nebuliser according to claim 16 wherein the restrictor is sized so as to ensure that the time taken to fill the control volume is around 1 ms.

19. A nebuliser according to claim 16 wherein the restrictor is sized so as to ensure that the time taken to fill the control volume is in the range 0.2.-0.3 s.

20. A nebuliser according to claim 1 wherein the output of the control valve is in fluid communication with a one-way valve, a sensing delay volume and a sensing delay valve which are adapted to delay the normal functioning of the sensing valve.

21. A nebuliser according to claim 6 wherein the sensing valve includes a restrictor which is sized such that the pressure acting on the sensing diaphragm required to seal the passageway is more positive than that required to open said passageway.
